(19) United States
(12) Patent Application Publication
(10) Pub. No.: US 2004/0243003 A1
(43) Pub. Date: Dec. 2, 2004

(54) Method and apparatus for bone diagnosis

(76) Inventors: Vladimir Pasternak, Bat-Yam (IL); Liat Tsoref, Tel-Aviv (IL); Michael Pasternak, Bat-Yam (IL); Andrey Mordvinov, Ramla (IL); Michael Martsinovsky, Rehovot (IL)

Correspondence Address: William H Dippert, Reed Smith, 29th Floor, 599 Lexington Avenue, New York, NY, 10022-7650, US

(21) Appl. No.: 10/484,399

(22) PCT Filed:
(86) PCT No.: PCT/IL02/00612

Related U.S. Application Data
(63) Continuation-in-part of application No. PCT/IL01/00683, filed on Jul. 24, 2001.

(51) Int. Cl. A61B 8/02

(52) U.S. Cl. 600/449

(57) ABSTRACT

A method for determining a value for at least one parameter that characterizes propagation of sound in a body part comprising: transmitting ultrasound pulses at each of a plurality of different distinct carrier frequencies through the body part; detecting the pulses after they are transmitted through the body part and generating signals responsive thereto; and processing the signals responsive to pulses at each of the carrier frequencies to determine a value for at least one parameter that characterizes propagation of ultrasound in the body part.

### RELATED APPLICATIONS

The present application is a continuation in part of PCT Application PCT/IL01/00683.

### FIELD OF THE INVENTION

The present invention relates to methods and apparatus for measuring characteristics of bone in-vivo and in particular for measuring characteristics of bone using ultrasound.

### BACKGROUND OF THE INVENTION

It is known to perform quantitative measurements of parameters that characterize the propagation of ultrasound in bone and to use the measurements to determine characteristics of bone and diagnose bone health. The performance of these measurements and their use in diagnosing the state and health of bone tissue is conventionally referred to as “quantitative ultrasound” (QUS). In particular, quantitative measurements of the speed of sound (SOS) in bone and attenuation of sound in bone as a function of frequency are used to diagnose the state and health of bone tissue. Attenuation of sound in bone is measured in dB and a dB change in attenuation per MHz change in frequency of ultrasound transmitted through bone and often per unit thickness of is used as a figure of merit in diagnosing bone tissue. The figure of merit is conventionally referred to as broadband ultrasound attenuation (BUA).

Typically, in QUS, values of SOS and BUA are used as statistical indicators of the state and health of a patient's bone tissue. Statistical correlation tables or normative databases that relate values of SOS and/or BUA with aspects of the state or health of bone and measurements of SOS and/or BUA acquired for the patient are used to diagnose the state or health of the patient's bone tissue. For example, values of SOS and BUA are used as statistical indicators of bone age, fracture probability, osteoporosis and osteopenia. PCT Application PCT/IL01/00683 entitled “Bone Age Assessment Using Ultrasound” and PCT Application PCT/IL01/00763 entitled “Joint Analysis Using Ultrasound”, the disclosures of which are incorporated herein by reference, discuss using QUS parameters to diagnose the state and/or health of bone tissue.

Measurements of SOS are often performed by sandwiching a part of the body, such as the heel, the wrist, or a finger, comprising an appropriate volume of bone between a pair of acoustic transducers. A first transducer of the pair transmits a pulse of ultrasound into the body part and a second transducer of the pair receives the transmitted pulse after it has passed through the body part. A transit time of the pulse through the body part and a distance between the first and second transducers is used to determine SOS in the bone comprised in the body part. To acquire BUA measurements a relatively short ultrasound pulse having a power spectrum characterized by a relatively broad spectral band is transmitted through the body part. A power spectrum of the pulse after transmission through the body part is compared to the power spectrum of the pulse prior to transmission to determine attenuation of ultrasound as a function of frequency. The power spectra are usually generated by processing the pulse shapes using an appropriate FFT algorithm.

Measurements of SOS and BUA acquired for a body part are sensitive to various parameters and ambient conditions that can affect accuracy and reliability of the measurements if not appropriately controlled. For example, SOS and BUA measurements are sensitive, inter alia, to acoustical contact of transducers used to acquire the measurements with the body part, ambient temperature and an amount of soft tissue in the body part.

BUA also appears to be sensitive to the shape of the bone in the body part relative to the orientation of the detectors. A waveform of an ultrasound pulse, after it is transmitted through a body part, is generally adulterated by reflections of acoustic energy in the pulse from features of the bone in the body part and interfaces of the bone with soft tissue. As a result, FFT analysis to determine a power spectrum for the waveform is generally limited to analysis of a relatively small leading portion of the waveform, which is relatively free of adulteration. The limitation to a small portion of the waveform reduces accuracy of the power spectrum determined for the pulse and thereby for a value of BUA for bone in the body part.

U.S. Pat. No. 6,095,979 varies a cross section size an ultrasonic beam transmitted through a body part to match the size of the cross section to the size of the body part to improve reliability of ultrasound bone diagnosis. For example, when performing bone assessment on small bones, the ultrasonic beam may be narrowed and its cross sectional area reduced to assure that substantially all the acoustic energy in the beam passes through a desired region of the bone.

U.S. Pat. No. 5,615,681 describes a method and apparatus for measuring the speed of sound in bone in which ultrasound transducers are pressed to a body part at a predetermined pressure. The predetermined pressure is “obtained by a transducer unit moving mechanism comprising a feed screw and a torque limiter”.

Japanese Patent H7-303643 describes an ultrasonic bone evaluation device comprising pressure sensors that sense pressure at which ultrasound transducers used to acquire QUS measurements for a body part are pressed to the body part. A controller dynamically controls force that presses the transducers to the body part responsive to pressure sensed by the pressure sensors to maintain a desired pressure at which the transducers press on the body part.

### SUMMARY OF THE PRESENT INVENTION

An aspect of some embodiments of the present invention relates to providing apparatus, hereinafter referred to as a “QUS monitor”, and methods for acquiring relatively accurate measurements of parameters that characterize the propagation of ultrasound in bone.

In some embodiments of the present invention, the propagation parameter is BUA. Alternatively or additionally, in some embodiments of the present invention, the propagation parameter is SOS.

An aspect of some embodiments of the present invention relates to providing a measure of the quality of acoustical coupling of an ultrasound transducer comprised in a QUS monitor that is coupled to a body part to determine a propagation parameter of ultrasound through the body part.

A QUS monitor, in accordance with an embodiment of the present invention, transmits ultrasound pulses through the body part at a plurality of different discrete carrier frequencies to acquire QUS measurements of the body part. Optionally ultrasound pulses at different carrier frequencies are transmitted through the body part at different times. Optionally, the QUS monitor utilizes a same first multi-frequency ultrasound transducer for transmitting ultrasound into the body part at all the different carrier frequencies. Optionally, the first transducer transmits pulses at different carrier frequencies into the body part via a same acoustic coupling-aperture of the first transducer that contacts the body part. Optionally, the QUS monitor utilizes a same second multi-frequency ultrasound transducer for receiving ultrasound pulses from the first transducer that are transmitted through the body part. Optionally the second transducer receives the transmitted pulses via a same acoustic coupling-aperture of the second transducer that contacts the body part.

An aspect of some embodiments of the present invention relates to providing an ultrasound transducer for transmitting ultrasound pulses at a plurality of distinct carrier frequencies through a body part for use in acquiring QUS measurements. An aspect of some embodiments of the present invention relates to providing an ultrasound transducer for sensing ultrasound pulses transmitted by the transmitting transducer through the body part at the distinct carrier frequencies. In accordance with an embodiment of the present invention, the transmitting transducer transmits ultrasound pulses at all of the carrier frequencies through a same acoustic-aperture, which is pressed to the body part. In accordance with an embodiment of the present invention, the sensing transducer receives ultrasound pulses at all the carrier frequencies that are transmitted through the body part through a same acoustic-aperture, which is pressed to the body part.

In accordance with an embodiment of the present invention, the QUS monitor determines attenuation of ultrasound pulses transmitted through the body part at different carrier frequencies and uses the determined attenuations to determine a value of BUA for bone in the body part. Optionally, the QUS monitor determines a value for BUA from at least one ratio between attenuation of the transmitted ultrasound pulses at different carrier frequencies.

The inventors have determined that a value for BUA for a body part determined from at least one attenuation ratio, in accordance with an embodiment of the present invention, generally provides a more accurate and reproducible value for BUA than is provided by prior art FFT analysis of wideband acoustic pulses transmitted through the body part. In addition, the measurements acquired in accordance with an embodiment of the present invention, are generally more independent of variations in bone shape than are BUA measurements provided by prior art methods. As a result measurements of BUA, in accordance with an embodiment of the present invention are generally more robust and reproducible than prior art measurements of BUA.

In accordance with an embodiment of the present invention, the QUS monitor determines values for SOS at different carrier frequencies. The determined values for SOS provide not only a value or values of SOS at a well-defined frequency or frequencies for use in diagnosing the state and health of bone tissue in the body part. In accordance with an embodiment of the present invention, measurements of SOS in the body part at the different characteristic frequencies are also used to determine a degree of dispersion in the speed of sound in the body part.

The inventors have determined that for a relatively wide range of frequencies that are typically used for QUS measurements, the speed of sound in bone comprised in a body part is relatively independent of frequency and does not exhibit substantial dispersion. In this “typical QUS range”, which extends from about 100 KHz to about 1 MHZ, presence of dispersion generally indicates that quality of acoustic coupling of an ultrasound transducer that is used to acquire QUS measurements for the body part is not satisfactory. In accordance with an embodiment of the present invention, if measurements of SOS in a body part at different carrier frequencies exhibits dispersion greater than a predetermined threshold dispersion, acoustic contact of an ultrasound transducer used to acquire QUS measurements is adjusted.

An aspect of some embodiments of the present invention relates to providing a new QUS parameter for use by a QUS monitor in diagnosing the state and health of bone tissue in a patient. In accordance with an embodiment of the present invention, the parameter is a function of a change in a characteristic time period of a pulse of ultrasound that the QUS monitor transmits through a body part that results from transmission of the pulse through the body part. The function is hereinafter referred to as a “time change coefficient” (TCC).

Optionally, the characteristic time period is a time lapse between a time at which the pulse is first detected by a suitable ultrasound transducer comprised in the QUS monitor after propagating through the body part and a subsequent first zero crossing of the pulse pressure detected by the transducer. The time lapse between the first detection time and the subsequent zero crossing time is hereinafter referred to as an “indicator period”. Optionally, TCC is a ratio between the indicator periods for ultrasound pulses at two carrier frequencies divided by a ratio of the indicator periods for pulses that are transmitted through a suitable phantom at the same carrier frequencies.

Bone tissue generally acts as a low pass filter for ultrasound pulses and attenuates higher frequency ultrasound components of an ultrasound pulse more than lower frequency components of the pulse. As a result, the indicator period of a pulse transmitted at a higher carrier frequency through a body part is generally lengthened by a greater factor than is an indicator period of a pulse transmitted through the body part at a lower carrier frequency. TCC is therefore correlated with BUA.

In accordance with an embodiment of the present invention, a new QUS parameter, is a function of a change in a time integral of the pressure amplitude of an ultrasound pulse that the QUS monitor transmits through a body part. The time integral is taken over a characteristic time period of the pulse of ultrasound and is equal to an area under that portion of a waveform representing the pressure amplitude, which is delimited by the characteristic period. The integral is hereinafter referred to as an indicator area and the parameter is hereinafter referred to as an area change coefficient (ACC). The ACC for a pulse is a function of the acoustic energy in that portion of the pulse defined by the characteristic time period.

Optionally, the characteristic time period is an indicator period. Optionally, ACC is a ratio between the indicator areas for ultrasound pulses at two carrier frequencies divided by a ratio of the indicator areas for pulses that are transmitted through a suitable phantom at the same carrier frequencies.

An indicator area of a pulse transmitted at a higher carrier frequency through a body part is generally decreased by a greater factor than is an indicator area of a pulse transmitted through the body part at a lower carrier frequency. (The decrease of the indicator area at the higher frequency relative to that at the lower frequency is caused by a relatively greater decrease in amplitude of the waveform at the higher frequency that offsets the relatively greater increase in the indicator period at the higher frequency.) ACC, as is TCC, is therefore also correlated with BUA.

There is therefore provided in accordance with an embodiment of the present invention, a method for determining a value for at least one parameter that characterizes propagation of sound in a body part comprising: transmitting ultrasound pulses at each of a plurality of different distinct carrier frequencies through the body part; detecting the pulses after they are transmitted through the body part and generating signals responsive thereto; and processing the signals responsive to pulses at each of the carrier frequencies to determine a value for at least one parameter that characterizes propagation of ultrasound in the body part.

Optionally the at least one parameter comprises a parameter that characterizes attenuation of ultrasound in the body part as a function of frequency. Optionally, the parameter is broadband ultrasound attenuation (BUA). Optionally, processing comprises determining attenuation in dB for at least one ultrasound pulse transmitted at each of the plurality of carrier frequencies and using the determined attenuations to determine a value for BUA.

The processing optionally comprises: determining a characteristic time period of a waveform of pulses transmitted through a phantom at each of the carrier frequencies after their propagation through the phantom; determining a characteristic frequency for pulses transmitted at each of the carrier frequencies responsive to the determined characteristic time periods; and using the characteristic frequencies to determine BUA.

Optionally, the plurality of carrier frequencies comprises a first carrier frequency and a higher second carrier frequency for which pulses transmitted by the first transducer have characteristic frequencies ν₁ and ν₂ and determining BUA comprises: determining amplitudes A₁ and A₂ of pulses transmitted at the first and second carrier frequencies after transmission through the body part; determining amplitudes Aₒ₂ and Aₒ₁ of the pulses transmitted at the first and second carrier frequencies prior to transmission through the body part; and determining BUA in accordance with the expression BUA=−[20log(A₂/Aₒ₂)−20log(A₁/Aₒ₁)]/[D(ν₂−ν₁)], where D is a path length of the pulses through the body part.

In some embodiments of the present invention, the at least one parameter comprises a time change coefficient (TCC) and processing comprises: determining first and second characteristic time periods of waveforms of pulses transmitted through the body part at respectively first and second carrier frequencies of the plurality of carrier frequencies; determining a first ratio between the first and second characteristic time periods; determining third and fourth time periods characteristic of the waveforms of pulses transmitted through a phantom at the first and second carrier frequencies; determining a second ratio between the third and fourth time periods; and determining a ratio between the first and second ratios to determine a value for TCC.

In some embodiments of the present invention, the at least one parameter comprises an area change coefficient (ACC) and processing comprises: determining first and second time integrals over characteristic time periods of waveforms of pulses transmitted through the body part at respectively first and second carrier frequencies of the plurality of carrier frequencies; determining a first ratio between the first and second time integrals; determining third and fourth time integrals over characteristic time periods of the waveforms of pulses transmitted through a phantom at the first and second carrier frequencies respectively; determining a second ratio between the third and fourth time integrals; and determining a ratio between the first and second ratios to determine a value for the ACC.

In some embodiments of the present invention, determining a characteristic time of the waveform of a pulse comprises: determining a first time at which the waveform is first detected; determining a second time at which a first subsequent zero crossing of the waveform occurs; and determining a difference between the first and second times.

In some embodiments of the present invention, transmitting ultrasound pulses comprises transmitting pulses at different carrier frequencies at different times.

There is further provided, in accordance with an embodiment of the present invention, apparatus for determining a value for at least one parameter that characterizes propagation of sound in a body part comprising: at least one first transducer acoustically coupled to the body part controllable to transmit ultrasound pulses at each of a plurality of different distinct carrier frequencies through the body part; at least one second transducer that is acoustically coupled to the body part, which detects pulses transmitted by the first transducer through the body part and generates signals responsive thereto; and a controller that receives signals generated by the second transducer responsive to transmitted pulses at each of the carrier frequencies and uses the signals to determine a value for at least one parameter that characterizes propagation of ultrasound in the body part.

Optionally, the at least one parameter comprises a parameter that characterizes attenuation of ultrasound in the body part as a function of frequency. Optionally, the parameter is broadband ultrasound attenuation (BUA).

The controller optionally, determines attenuation in dB for at least one ultrasound pulse transmitted at each of the plurality of carrier frequencies and uses the determined attenuation to determine a value for BUA. Optionally, the controller determines a characteristic frequency for pulses transmitted at each of the plurality of carrier frequencies responsive to a characteristic time period of a waveform of pulses transmitted by the first transducer through a phantom at the frequency and uses the characteristic frequencies to determine BUA.

Optionally, the plurality of carrier frequencies comprises a first carrier frequency and a higher second carrier frequency for which pulses transmitted by the first transducer have characteristic frequencies ν₁ and ν₂ respectively and wherein BUA for the body part is determined in accordance with the expression BUA=−[20log(A₂/Aₒ₂)−20log(A₁/Aₒ₁)]/[D(ν₂−ν₁)], where A₂ and A₁ are the amplitudes of pulses transmitted by the first transducer at the higher and lower frequencies respectively after their transmission through the body Part, Aₒ₂ and Aₒ₁ are amplitudes of the pulses prior to transmission through the body part and D is a distance between a first and a second transducer.

In some embodiments of the present invention, the at least one parameter comprises a time change coefficient (TCC) which the controller determines by: determining first and second characteristic time periods of waveforms of pulses transmitted through the body part at respectively first and second carrier frequencies of the plurality of carrier frequencies; determining a first ratio between the first and second characteristic time periods; determining third and fourth time periods characteristic of the waveforms of pulses transmitted through a phantom at the first and second carrier frequencies; determining a second ratio between the third and fourth time periods; and determining a ratio between the first and second ratios to determine a value for TCC.

In some embodiments of the present invention, the at least one parameter comprises an area change coefficient (ACC) which the controller determines by: determining first and second time integrals over characteristic time periods of waveforms of pulses transmitted through the body part at respectively first and second carrier frequencies of the plurality of carrier frequencies; determining a first ratio between the first and second time integrals; determining third and fourth time integrals over characteristic time periods of the waveforms of pulses transmitted through a phantom at the first and second carrier frequencies respectively; determining a second ratio between the third and fourth time integrals; and determining a ratio between the first and second ratios to determine a value for the ACC.

In some embodiments of the present invention, the at least one parameter comprises the speed of sound (SOS) in the body part. Optionally, the controller uses SOSs determined for at least two of the carrier frequencies to determine dispersion of the speed of sound in the body part.

Optionally, for SOS determined for each carrier frequency the controller determines a characteristic frequency which is proportional to an inverse of a characteristic time period of a waveform of pulses transmitted by the first transducer through a phantom that are sensed by the second transducer and wherein the controller determines dispersion as a function of the characteristic frequencies.

Additionally or alternatively, the controller determines quality of acoustic coupling to the body part of the at least one first transducer and the at least one second transducer responsive to the determined dispersion.

Optionally, the controller compares the determined dispersion to a predetermined threshold dispersion and if the determined dispersion is greater than the threshold dispersion, the controller determines that quality of coupling is not acceptable for determining a value of the at least one parameter.

Optionally, the threshold dispersion is greater than or equal to 75 m/s per MHz. Alternatively, the threshold dispersion is optionally greater than or equal to 150 m/s per MHz.

In some embodiments of the present invention, at least one of the first and second transducers is controllable to be moved by the controller and the controller controls motion of the at least one transducer to improve the acoustic coupling if quality of acoustic coupling is not acceptable.

In some embodiments of the present invention, the body part is positioned on a pedestal controllable to be moved by the controller relative to the at least one first and at least one second transducer and if quality of acoustic coupling is not acceptable, the controller controls motion of the pedestal to improve the acoustic coupling.

In some embodiments of the present invention, the characteristic time period of the waveform of the pulse is a time period between a time at which the pulse is first detected by the at least one second transducer and a first subsequent zero crossing of the pulse pressure detected by the transducer.

In some embodiments of the present invention, the at least one first transducer comprises a single multi-frequency first transducer controllable to transmit pulses at each of the plurality of carrier frequencies. In some embodiments of the present invention, the at least one second transducer comprises a single multi-frequency second transducer sensitive to pulses transmitted at each of the carrier frequencies.

In some embodiments of the present invention, the multi-frequency transducer comprises: a separate piezoelectric vibrator having a longitudinal axis and two planar face surfaces perpendicular thereto for each carrier frequency, said vibrator having a resonant frequency substantially equal to the carrier frequency; and at least one separate electrical isolation section having a longitudinal axis and two planar face surfaces perpendicular thereto; wherein the vibrators and isolation sections are aligned with their respective longitudinal axes substantially collinear and bonded together with an electrical isolation section sandwiched between every two vibrators to form a single mechanically integral stack.

Optionally, each isolation section comprises a conducting layer sandwiched between two plates formed from a non-polarized piezoelectric material. Alternatively or additionally, the stack is bonded to an acoustic damper at one end of the stack.

In some embodiments of the present invention, the piezoelectric material or materials from which the vibrators and isolation sections are formed have substantially same acoustic impedance.

In some embodiments of the present invention, the stack is mounted in a housing formed from a conducting material.

In some embodiments of the present invention, the plurality of carrier frequencies comprises a pair of frequencies that straddle a range of frequencies for which bone tissue typically attenuates ultrasound by about 3 dB.

Optionally, the lower frequency of the pair of frequencies is less than about 150 KHz. Optionally, the lower frequency is substantially equal to about 125 KHz.

In some embodiments of the present invention, the upper frequency of the pair of frequencies is greater than about 300 KHz. Optionally, the upper frequency is substantially equal to about 750 KHz.

In some embodiments of the present invention, the controller controls the first transducer to transmit pulses at different carrier frequencies at different times.

### BRIEF DESCRIPTION OF FIGURES

Non-limiting examples of embodiments of the present invention are described below with reference to figures attached hereto and listed below. In the figures, identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1A and 1B schematically show a QUS monitor acquiring QUS measurements of a body part at two different ultrasound carrier frequencies, in accordance with an embodiment of the present invention;

FIGS. 2A and 2B schematically show waveforms of ultrasound pulses transmitted by the QUS monitor shown in FIGS. 1A and 1B through a phantom, in accordance with an embodiment of the present invention; and

FIG. 3 schematically shows a multi-frequency ultrasound transducer, in accordance with an embodiment of the present invention.

### DETAILED DESCRIPTION OF EXEMPLARY EMBODIMENTS

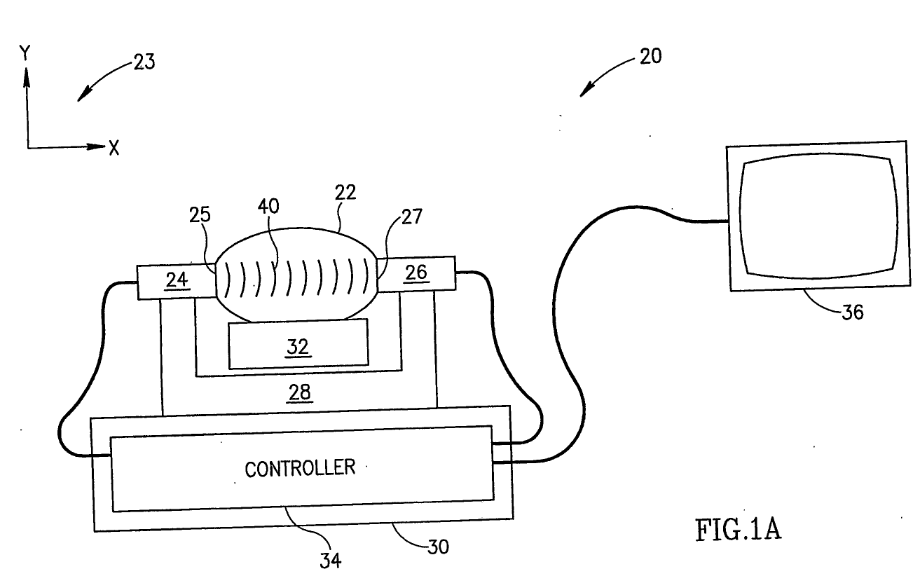

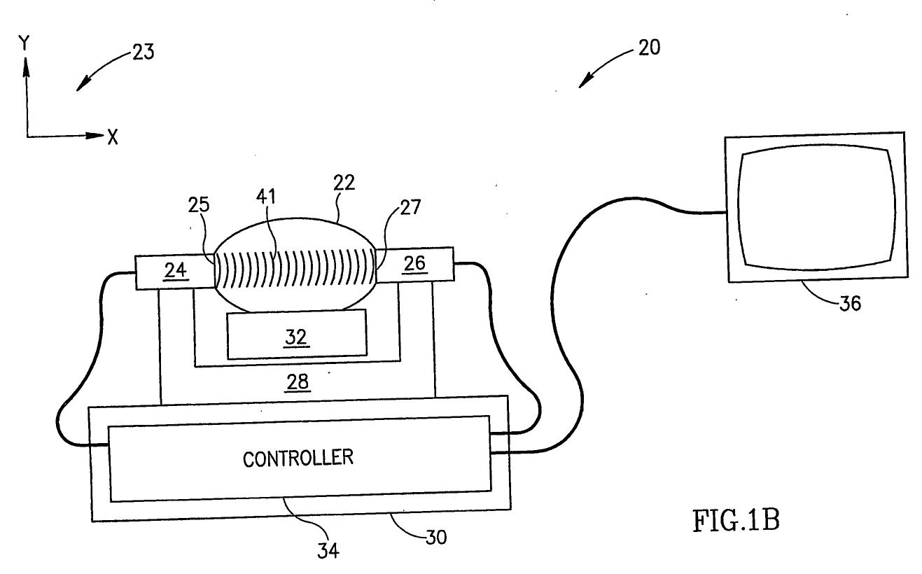

FIGS. 1A and 1B show schematic cross sectional views of a QUS monitor 20 acquiring QUS measurements of a body part 22 of a patient (not shown) to diagnose the state and or health of bone in the body part. Body part 22 may, by way of example be the patient's finger, with QUS measurements acquired for the phalanges; the patient's wrist, with QUS measurements acquired for carpal bones or radius and ulna bones; or the patient's heel, with QUS measurements acquired for the calcaneous. By way of example, in FIGS. 1A and 1B body part 22 is assumed to be the patient's wrist and the cross section in the figure is a schematic cross-section of the wrist. For convenience of presentation a coordinate system 23 having x and y-axes is used to reference locations of features of QUS monitor 20 and directions pertinent to the description of the monitor.

QUS monitor 20 comprises a multi-frequency transducer 24 for transmitting ultrasound pulses through body part 22 at a plurality of different distinct ultrasound carrier frequencies and a multi-frequency transducer 26 for sensing the ultrasound pulses transmitted by transducer 24 at each of the carrier frequencies. Transmitting transducer 24 optionally transmits ultrasound pulses at all of the carrier frequencies into body part 22 through a same acoustic aperture (indicated at reference 25) of the transducer, which is pressed to the body part. Sensing transducer 26 optionally receives ultrasound transmitted by transmitting transducer 24 at all of the carrier frequencies through a same acoustic aperture (indicated at reference 27) of the transducer, which is pressed to the body part. Optionally, the frequency response of sensing transducer 26 is tuned to the frequency spectrum of pulses transmitted at each of the carrier frequencies after the pulses have been transmitted through bone and have had their frequency spectrum modified by attenuation that is typical of that caused by bone tissue.

Transducers 24 and 26 are optionally mounted to a same bracket 28 so that they can be moved towards each other along the x-axis to firmly press on body part 22 and be moved away from each other to release the body part. Optionally, a pressure sensor (not shown) is coupled to at least one of transducers 24 and 26 to sense pressure with which the transducers press on body part 22. Optionally, one of transducers 24 and 26 is fixed to bracket 28 and the other is moveable back and forth along the x-axis to move the transducers towards or away from each other. Optionally, motion of the moveable transducer is controlled responsive to signals generated by the pressure sensor so that transducers 24 and 26 press on body part 22 with a desired pressure.

Bracket 28 is optionally mounted to a base 30 having a support pedestal 32 mounted thereto that supports body part 22. Optionally, bracket 28 is free to move along the x-direction so that motion of body part 22 along the x-direction during acquisition of QUS measurements does not generate substantial differences in pressure with which transducers 24 and 26 press on the body part. Motion of body part 22 during acquisition of QUS measurements may for example be generated by inadvertent motion of the patient.

Optionally, pedestal 32 is moveable up or down along the y direction relative to transducers 24 and 26 so that the pedestal can be positioned to properly locate body part 22 between transducers 24 and 26. In some embodiments of the present invention, pedestal 32 is moveable along a z-direction, which is perpendicular to the x and y-axes, to position body part 22 between transducers 24 and 26. Any of various and many methods and devices known in the art may be used to mount transducers 24 and 26 to bracket 28, the bracket to base 30 and pedestal 32 to the base.

A controller 34 controls transmitting transducer 24 to transmit pulses of ultrasound through body part 22 at each of the different carrier frequencies, optionally, sequentially. Signals generated by sensing transducer 26 responsive to pulses transmitted through body part 22 by transmitter 24 are transmitted to controller 34. Controller 34 processes signals it receives generated by sensing transducer 26 responsive to the pulses at the different carrier frequencies to determine values for QUS parameters useable to assess the state and/or health of bone in body part 22. Optionally controller 34 displays the parameters and their diagnostic content in suitable formats on a console 36.

By way of example, it is assumed that transmitting transducer 24 is controllable by controller 34 to transmit ultrasound at two carrier frequencies. In accordance with an embodiment of the present invention, one of the two carrier frequencies is a relatively low frequency and the other of the two frequencies is a relatively high frequency.

As noted above, bone tissue acts as a low pass filter for ultrasound and typically attenuates ultrasound by about 3 dB at a “cutoff” frequency in a range of frequencies from about 150 KHz to about 300 KHz. Optionally, the low and high carrier frequencies are chosen so that they straddle the 3 dB cutoff range of frequencies. Optionally, the low carrier frequency is less than about 150 KHz. Optionally, the high carrier frequency is greater than about 300 KHz. In some embodiments of the present invention, the low carrier frequency is about 125 KHz. In some embodiments of the present invention the high carrier frequency is about 750 KHz.

FIG. 1A schematically shows controller 34 exciting transmitting transducer 24 to transmit an ultrasound pulse indicated by curved lines 40 through body part 22 at the low frequency. The low frequency of the low frequency pulse is indicated schematically by the relatively large spacing between lines 40. FIG. 1B schematically shows controller 34 exciting transmitting transducer 24 to transmit a high frequency pulse indicted by curved lines 41. The high frequency of the high frequency pulse is indicated schematically by the relatively small spacing between lines 41.

In accordance with an embodiment of the present invention, controller 34 determines a value for BUA for body part 22 from the amplitudes of ultrasound pulses 40 and 41 sensed by sensing transducer 26 and a carrier frequency characteristic of each of the pulses. Whereas pulses 40 and 41 are generated by exciting transducer 24 nominally at the high and low carrier frequencies respectively, the actual carrier frequencies that characterize pulses 40 and 41 may differ from their respective nominal carrier frequencies. A difference between an actual “characteristic” carrier frequency of pulse 40 or pulse 41 and its nominal carrier frequency may for example be generated by drift in an element of transducer 24 or controller 34 caused by changes in the ambient environment of QUS monitor 20. As a result, in accordance with an embodiment of the present invention, the characteristic carrier frequencies of pulses 40 and 41 are determined, as discussed below, from the waveform of pulses transmitted at the high and low frequencies through a suitable phantom.

Let the amplitude of high frequency and low frequency pulses 40 and 41 sensed by sensing transducer 26 be represented by A_{H} and A_{L} respectively and let the characteristic carrier frequencies of the pulses be represented respectively by ν_{H} and ν_{L}. Let the amplitudes of pulses 40 and 41 transmitted by transmitting transducer 24 immediately prior to entrance into body part 22 be represented by A_{oH} and A_{oL} respectively and a distance between transmitting transducer24 and sensing transducer 26 be “D”. Distance D may be determined using any of many various methods and devices known in the art, such as for example a digital linear encoder.

Attenuation of high carrier frequency pulse 41 in dB, which results from transmission through body part 22 and losses in sensing transducer 26, is equal to 20log(A_{H}/A_{oH}). Similarly attenuation of low carrier frequency pulse 40 is equal to 20log(A_{L}/A_{oL}). In accordance with an embodiment of the present invention, BUA for body part 22 is determined in accordance with the expression BUA=−[20log(A_{H}/A_{oH})−20log(A_{L}/A_{oL})]/[D(ν_{H}−ν_{L})]. Rearranging the expression, BUA may be written BUA=−[20log(A_{H}/A_{L})−20log(A_{oH}/A_{oL})]/[D(ν_{H}−ν_{L})]. A_{oH}/A_{oL} is optionally determined by calibration of QUS monitor 20 using an appropriate phantom and any of various methods and devices known in the art. Optionally, the phantom is formed from a material, such as Perspex, that attenuates substantially all frequency components of pulses transmitted at the nominal high and low carrier frequencies by substantially a same amount.

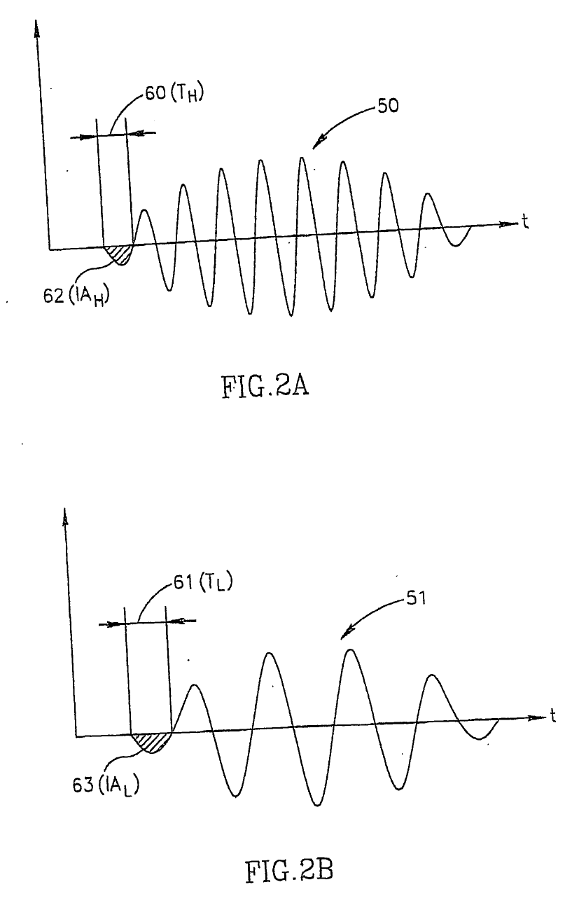

In accordance with an embodiment of the present invention, ν_{H} and ν_{L}, as noted above, are determined from the waveforms of pulses transmitted by transmitting transducer 24 through a suitable phantom at the nominal high and low carrier frequencies. In accordance with an embodiment of the present invention, a characteristic carrier frequency of a pulse transmitted through the phantom is determined to be equal to an inverse of a time period that characterizes the transmitted pulse. Optionally, the characteristic time period, i.e. an “indicator” time period, is equal to a time lapse between a time at which the pulse is first detected by sensing transducer 26 and a subsequent first zero crossing of the pulse pressure detected by the transducer. Schematic waveforms 50 and 51 as functions of time for pulses transmitted through the phantom at the nominal high and low frequencies and their respective indicator periods 60 and 61 are shown in FIGS. 2A and 2B respectively.

Let the durations of indicator periods 60 and 61 be represented by times T_{H} and T_{L} respectively. Indicator periods 60 and 61 are “half wavelength periods” generated by frequency components of the pulse having frequencies substantially equal to 1/(2T_{H}) and 1/(2T_{L}) respectively. In accordance with an embodiment of the present invention, the characteristic frequency ν_{H}=1/(2T_{H}) and characteristic frequency ν_{L}=1/(2T_{L}). Introducing the expressions for ν_{H} and ν_{L} into the expression for BUA given above, BUA in accordance with an embodiment of the present invention, may be written, BUA=−[40log(A_{H}/A_{L})−40log(A_{oH}/A_{oL})]/[D(1/T_{H}−1/T_{L})].

In some embodiments of the present invention a new QUS indicator, referred to as the time change coefficient TCC, is used to indicate the state and/or health of bone tissue. The inventors have determined that the duration of an indicator period for a pulse transmitted through a body part at the nominal high or low carrier frequencies after transmission through the body part may be different from that of a pulse at the same carrier frequency transmitted through the phantom. In particular, as a result of bone tissue attenuating higher frequency ultrasound waves substantially more than lower frequency sound waves, the indicator period of the pulse transmitted through the body part is generally lengthened relative to that of the pulse transmitted through the phantom. (It is assumed that the phantom attenuates ultrasound frequencies in the bandwidths of the high and low carrier frequency pulse substantially equally.) In addition, in general, to an extent that the bone tissue exhibits a greater degree of BUA, a difference between the lengthening of the indicator period of a high frequency pulse transmitted through the body part is greater than that of a lower frequency pulse transmitted through the body part. The time change coefficient TCC, is defined, in accordance with an embodiment of the present invention, as a function of the indicator periods of pulses at two different carrier frequencies transmitted through a body part. The TCC for the body part is responsive to changes in the indicator periods of the pulses and thereby to attenuation of ultrasound in bone tissue of the body part.

For QUS monitor 20 shown in FIGS. 1A and 1B, let the durations of indicator periods of high and low frequency pulses 40 and 41 after transmission through body part 22 be represented by T′_{H} and T′_{L} respectively. The time change coefficient TCC for the pulses is optionally defined as (T_{L}/T_{H})/(T′_{L}/T′_{H}). It is noted that for the definition of TCC given in the preceding sentence the value of TCC decreases as the value of BUA for body part 22 increases.

In some embodiments of the present invention the new QUS indicator, referred to as the area change coefficient ACC, is used to indicate the state and/or health of bone tissue. As noted above, ACC is a function of a change in a time integral of the pressure amplitude of an ultrasound pulse that the QUS monitor transmits through a body part. The time integral is taken over a characteristic time period of the pulse of ultrasound and is equal to an area, i.e. the indicator area, under that portion of a waveform representing the pressure amplitude, which is delimited by the characteristic period. Optionally, the characteristic time period is an indicator period. Optionally, ACC is a ratio between the indicator areas for ultrasound pulses at two carrier frequencies divided by a ratio of the indicator areas for pulses that are transmitted through a suitable phantom at the same carrier frequencies.

Indicator areas 62 and 63 for high and low frequency waveforms 50 and 51 corresponding to indicator periods 60 and 61 are shown shaded in FIGS. 2A and 2B. Let the areas of indicator areas 62 and 63 be represented respectively by IA_{H} and IA_{L}. Let indicator areas of high and low frequency pulses 40 and 41 after transmission through body part 22 be represented by IA′_{H} and IA′_{L} respectively. The area change coefficient ACC for the pulses is optionally defined as (IA_{L}/IA_{H})/(IA′_{L}/IA′_{H}). It is noted that for the definition of ACC given in the preceding sentence, the value of ACC increases as the value of BUA for body part 22 increases.

In some embodiments of the present invention QUS monitor 20 determines the speed of sound (SOS) for pulses transmitted through body part 22 at the high carrier frequency and at the low carrier frequency. The speed of sound at a carrier frequency of transmitting transducer 24 is determined from the distance D between transducers 24 and 26 and the transit time through body part 22 of at least one pulse transmitted by the transducer at the carrier frequency. The determined values of SOS are used as QUS parameters for determining the state and/or health of bone tissue in body part 22.

In some embodiments of the present invention controller 34 determines if measurements of SOS at the high and low frequencies indicate that the speed of sound in body part 22 evidences dispersion. The inventors have determined that dispersion in measurements of SOS for a body part is generally not generated by bone tissue in the body part. Generally, dispersion in measurements of SOS indicates that acoustic coupling of an ultrasound transducer used to acquire the SOS measurements is compromised. Therefore, in accordance with an embodiment of the present invention, if controller 34 determines that measurements of SOS indicate dispersion greater than a predetermined dispersion threshold, the controller adjusts the coupling of transducers 24 and 26 to body part 22 to reduce the dispersion and improve thereby the acoustic coupling of the transducers to the body part.

In some embodiments of the present invention, the predetermined dispersion threshold is greater than or equal to 75 m/s per MHz. In some embodiments of the present invention, the predetermined dispersion threshold is greater than or equal to 150 m/s per MHz. In accordance with an embodiment of the present invention, a frequency at which an SOS measurement is made for a body part is defined to be a frequency determined responsive to an indicator period of a pulse that is transmitted through the body part to determine the SOS. The “per MHz” units of the dispersion thresholds given above therefore refer to a “per MHz” determined form the “indicator period” frequencies at which the SOS measurements are acquired.

In some embodiments of the present invention, controller 34 adjusts a pressure at which transducers 24 and 26 press on body part 22 to substantially minimize dispersion In some embodiments of the present invention controller 34 adjusts the y-coordinate and/or the z-coordinate of pedestal 32 to substantially minimize dispersion.

In accordance with an embodiment of the present invention, controller 34 determines if SOS measurements evidence dispersion and adjust acoustic coupling of transducers 24 and 26 prior to acquiring QUS measurements for use in determining state or health of bone in body part 22. In some embodiments of the present invention controller 34 determines SOS dispersion and adjusts acoustic coupling of transducers 24 and 26 responsive thereto periodically during acquisition of QUS measurement for body part 22.

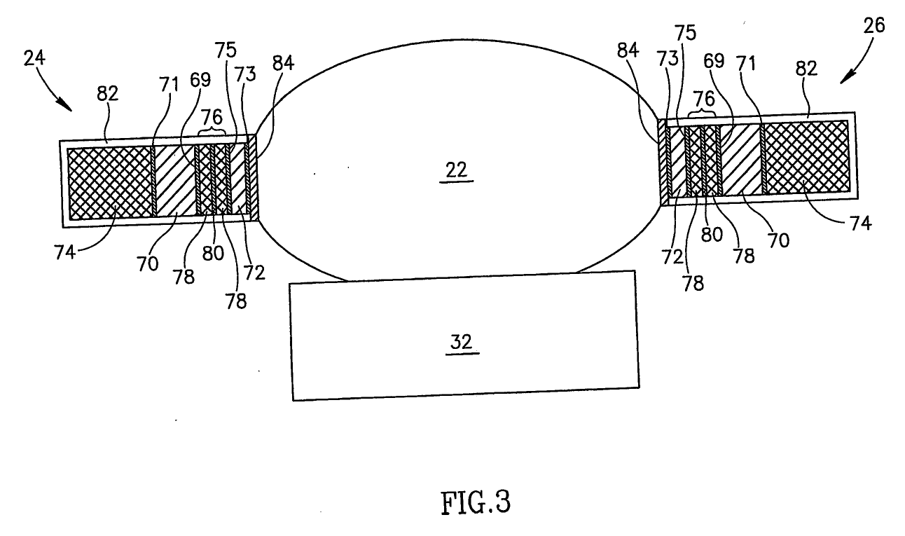

FIG. 3 shows enlarged schematic cross-sections of transmitting transducer 24 and sensing transducer 26 shown in FIGS. 1A and 1B, which show design and components of the transducers, in accordance with an embodiment of the present invention.

Multi-frequency transmitting transducer 24 comprises a low frequency piezoelectric vibrator 70, a high frequency piezoelectric vibrator 72 and an acoustic damper 74. High and low frequency vibrators 70 and 72 are formed from suitable piezoelectric materials having a same acoustic impedance and optionally from a same piezoelectric material. Dimensions of high and low frequency vibrators 70 and 72 are determined so that they have desired high and low resonant frequencies respectively. High frequency vibrator 70 has electrodes 69 and 71 mounted thereon and low frequency vibrator 72 has electrodes 73 and 75 mounted thereon.

The high and low frequency vibrators are separated by an isolation section 76 that electrically isolates vibrator 70 from vibrator 72. Isolation section 76 comprises two plates 78 of material bonded together with a conductive layer 80 between them. Conductive layer 80 is, optionally, electrically connected to housing 82. The material from which plates 78 are formed has acoustic impedance that is substantially the same as that of the material from which vibrators 70 and 72 are formed. Optionally, plates 78 are formed from the same material that vibrators 70 and 72 are formed, but whereas the material in vibrators 70 and 72 is polarized, the material in plates 78 is un-polarized.

Vibrators 70 and 72, isolation section 76 and acoustic damper 74 are bonded together using a suitable epoxy to form an integral “acoustic stack”. Optionally, vibrators 70 and 72, isolation section 74 and acoustic damper 78 are bonded together under pressure so that layers of epoxy that bonds them together are thin. The acoustic stack is mounted in a conductive housing 82 formed from a metal such as aluminum. A cap plate 84, optionally formed from a material having an acoustic impedance intermediate that of vibrator 72 and the human body, closes the acoustic stack inside housing 82. For many piezoelectric materials from which vibrator 72 may be formed a polycarbonate has suitable impedance advantageous for the practice of the present invention. Cap 84 functions as an acoustic aperture for transmitting transducer 24 through which ultrasound pulses are transmitted to body part 22.

By way of a numerical example, in some embodiments of the present invention, transmitting transducer 24 is configured to transmit ultrasound pulses at a low carrier frequency of about 125 KHz and a high carrier frequency of about 750 KHz. Components of the transmitting transducer inside housing 82, e.g. vibrators 70, 72, isolation section 76, optionally, all have a circular cross section of about 20 mm so the acoustic stack that they form when bonded together is cylindrical in shape and has a diameter of about 20 mm. High frequency vibrator 72 has a resonant frequency of about 750 KHz and a corresponding thickness of about 1.5 mm. Low frequency vibrator 70 has a resonant frequency of about 125 KHz and a corresponding thickness of about 9 mm. Low frequency vibrator 70 may be formed by bonding together six high frequency vibrators 72 so that they are acoustically in series and electrically in parallel.

It is noted that whereas transducer 24 comprises two vibrators and provides ultrasound pulses at two distinct carrier frequencies, a similar construction can be used to provide an ultrasound transducer that provides pulses at more than two carrier frequencies. For example for each additional frequency desired, an additional vibrator having a resonant frequency substantially equal to that of the additional frequency is added to the acoustic stack of the vibrator. Each additional vibrator is electrically isolated from other vibrators in the stack by an isolation section similar to isolation section 76.

A power supply (not shown) generates vibrations in high and low frequency vibrators 70 and 72 to generate high and low frequency ultrasound pulses for acquiring QUS measurements by appropriately electrifying excitation electrodes 69 and 71 and 73 and 75 respectively.

Sensing transducer 26 is optionally identical to transmitting transducer 24. In some embodiments of the present invention sensing 26 is identical to transmitting transducer 24 except for cap 84, which in the sensing transducer is formed from a conductor to improve electrical shielding of electrodes

In the description and claims of the present application, each of the verbs, “comprise” “include” and “have”, and conjugates thereof, are used to indicate that the object or objects of the verb are not necessarily an exhaustive listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. A method for determining a value for at least one parameter that characterizes propagation of sound in a body part comprising:
transmitting ultrasound pulses at each of a plurality of different distinct carrier frequencies through the body part;
detecting the pulses after they are transmitted through the body part and generating signals responsive thereto; and
processing the signals responsive to pulses at each of the carrier frequencies to determine a value for at least one parameter that characterizes propagation of ultrasound in the body part.

2. A method according to claim 1 wherein the at least one parameter comprises a parameter that characterizes attenuation of ultrasound its the body part as a function of frequency.

3. A method according to claim 2 wherein the parameter is broadband ultrasound attenuation (BUA).

4. A method according to claim 3 wherein processing comprises determining attenuation in dB for at least one ultrasound pulse transmitted at each of the plurality of carrier frequencies and using the determined attenuations to determine a value for BUA.

5. A method according to claim 4 wherein processing comprises:
determining a characteristic time period of a waveform of pulses transmitted through a phantom at each of the carrier frequencies after their propagation through the phantom;
determining a characteristic frequency for pulses transmitted at each of the carrier frequencies responsive to the determined characteristic time periods; and
using the characteristic frequencies to determine BUA.

6. A method according to claim 5 wherein the plurality of carrier frequencies comprises a first carrier frequency and a higher second carrier frequency for which pulses transmitted by the first transducer have characteristic frequencies ν₁ and ν₂ and determining BUA comprises:
determining amplitudes A₁ and A₂ of pulses transmitted at the first and second carrier frequencies after transmission through the body part;
determining amplitudes Aₒ₂ and Aₒ₁ of the pulses transmitted at the first and second carrier frequencies prior to transmission through the body part; and
determining BUA in accordance with the expression BUA=−[20log(A₂/oₒ₂)−20log(A₁/Aₒ₁)]/[D(ν₂−ν₁)], where D is a path length of the pulses through the body part.

7. A method according to claim 1 wherein the at least one parameter comprises a time change coefficient (TCC) and processing comprises:
determining first and second characteristic time periods of waveforms of pulses transmitted through the body part at respectively first and second carrier frequencies of the plurality of carrier frequencies;
determining a first ratio between the first and second characteristic time periods;
determining third and fourth time periods characteristic of the waveforms of pulses transmitted through a phantom at the first and second carrier frequencies; determining a second ratio between the third and fourth time periods; and
determining a ratio between the first and second ratios to determine a value for TCC.

8. A method according to claim 1 wherein the at least one parameter comprises an area change coefficient (ACC) and processing comprises:
determining first and second time integrals over characteristic time periods of waveforms of pulses transmitted through the body part at respectively first and second carrier frequencies of the plurality of carrier frequencies;
determining a first ratio between the first and second time integrals;
determining third and fourth time integrals over characteristic time periods of the waveforms of pulses transmitted through a phantom at the first and second carrier frequencies respectively;
determining a second ratio between the third and fourth time integrals; and
determining a ratio between the first and second ratios to determine a value for the ACC.

9. A method according to claim 5 wherein determining a characteristic time period of the waveform of a pulse comprises:
determining a first time at which the waveform is first detected;
determining a second time at which a first subsequent zero crossing of the waveform occurs; and
determining a difference between the first and second times.

10. Apparatus for determining a value for at least one parameter that characterizes propagation of sound in a body part comprising:
at least one first transducer acoustically coupled to the body part controllable to transmit ultrasound pulses at each of a plurality of different distinct carrier frequencies through the body part;
at least one second transducer that is acoustically coupled to the body part, which detects pulses transmitted by the first transducer through the body part and generates signals responsive thereto; and
a controller that receives signals generated by the second transducer responsive to transmitted pulses at each of the carrier frequencies and uses the signals to determine a value for at least one parameter that characterizes propagation of ultrasound in the body part.

11. Apparatus according to claim 10 wherein the at least one parameter comprises a parameter that characterizes attenuation of ultrasound in the body part as a function of frequency.

12. Apparatus according to claim 11 wherein the parameter is broadband ultrasound attenuation (BUA)

13. Apparatus according to claim 12 wherein the controller determines attenuation in dB for at least one ultrasound pulse transmitted at each of the plurality of carrier frequencies and uses the determined attenuation to determine a value for BUA.

14. Apparatus according to claim 13 wherein the controller determines a characteristic frequency for pulses transmitted at each of the plurality of carrier frequencies responsive to a characteristic time period of a waveform of pulses transmitted by the first transducer through a phantom at the frequency and uses the characteristic frequencies to determine BUA.

15. Apparatus according to claim 14 wherein the plurality of carrier frequencies comprises a first carrier frequency and a higher second carrier frequency for which pulses transmitted by the first transducer have characteristic frequencies ν₁ and ν₂ respectively and wherein BUA for the body part is determined in accordance with the expression BUA=−[20log(A₂/Aₒ₂)−20log(A₁/Aₒ₁)]/[D(ν₂−ν₁)], where A₂ and A₁ are the amplitudes of pulses transmitted by the first transducer at the higher and lower frequencies respectively after their transmission through the body part Aₒ₂ and Aₒ₁ are amplitudes of the pulses prior to transmission through the body part and D is a distance between a first and a second transducer.

16. Apparatus according to claim 10 wherein the at least one parameter comprises a time change coefficient (TCC) which the controller determines by:
determining first and second characteristic time periods of waveforms of pulses transmitted through the body part at respectively first and second carrier frequencies of the plurality of carrier frequencies;
determining a first ratio between the first and second characteristic time periods;
determining third and fourth time periods characteristic of the waveforms of pulses transmitted through a phantom at the first and second carrier frequencies; determining a second ratio between the third and fourth time periods; and
determining a ratio between the first and second ratios to determine a value for TCC.

17. Apparatus according to claim 10 wherein the at least one parameter comprises an area change coefficient (ACC) which the controller determines by:
determining first and second time integrals over characteristic time periods of waveforms of pulses transmitted through the body part at respectively first and second carrier frequencies of the plurality of carrier frequencies;
determining a first ratio between the first and second time integrals;
determining third and fourth time integrals over characteristic time periods of the waveforms of pulses transmitted through a phantom at the first and second carrier frequencies respectively;
determining a second ratio between the third and fourth time integrals; and
determining a ratio between the first and second ratios to determine a value for the ACC.

18. Apparatus according to claim 10 wherein the at least one parameter comprises the speed of sound (SOS) in the body part.

19. Apparatus according to claim 18 wherein the controller uses SOSs determined for at least two of the carrier frequencies to determine dispersion of the speed of sound in the body part.

20. Apparatus according to claim 19 wherein for SOS determined for each carrier frequency the controller determines a characteristic frequency which is proportional to an inverse of a characteristic time period of a waveform of pulses transmitted by the first transducer through a phantom that are sensed by the second transducer and wherein the controller determines dispersion as a function of the characteristic frequencies.

21. Apparatus according to claim 19 wherein the controller determines quality of acoustic coupling to the body part of the at least one first transducer and the at least one second transducer responsive to the determined dispersion.

22. Apparatus according to claim 21 wherein the controller compares the determined dispersion to a predetermined threshold dispersion and if the determined dispersion is greater than the threshold dispersion, the controller determines that quality of coupling is not acceptable for determining a value of the at least one parameter.

23. Apparatus according to claim 22 wherein the threshold dispersion is greater than or equal to 75 m/s per MHz.

24. Apparatus according to claim 22 wherein the threshold dispersion is greater than or equal to 150 m/s per MHz.

25. Apparatus according to claim 22 wherein at least one of the first and second transducers is controllable to be moved by the controller and the controller controls motion of the at least one transducer to improve the acoustic coupling if quality of acoustic coupling is not acceptable.

26. Apparatus according to claim 22 wherein the body part is positioned on a pedestal controllable to be moved by the controller relative to the at least one first and at least one second transducer and if quality of acoustic coupling is not acceptable, the controller controls motion of the pedestal to improve the acoustic coupling.

27. Apparatus according to claim 14 wherein the characteristic time period of the waveform of the pulse is a time period between a time at which the pulse is first detected by the at least one second transducer and a first subsequent zero crossing of the pulse pressure detected by the transducer.

28. Apparatus according to claim 10 wherein the at least one first transducer comprises a single multi-frequency first transducer controllable to transmit pulses at each of the plurality of carrier frequencies.

29. Apparatus according to claim 10 wherein the at least one second transducer comprises a single multi-frequency second transducer sensitive to pulses transmitted at each of the carrier frequencies.

30. Apparatus according to claim 28 wherein a multi-frequency transducer comprises:
a separate piezoelectric vibrator having a longitudinal axis and two planar face surfaces perpendicular thereto for each carrier frequency, said vibrator having a resonant frequency substantially equal to the carrier frequency; and
at least one separate electrical isolation section having a longitudinal axis and two planar face surfaces perpendicular thereto;
wherein the vibrators and isolation sections are aligned with their respective longitudinal axes substantially collinear and bonded together with an electrical isolation section sandwiched between every two vibrators to form a single mechanically integral stack.

31. Apparatus according to claim 30 wherein each isolation section comprises a conducting layer sandwiched between two plates formed from a non-polarized piezoelectric material.

32. Apparatus according to claim 30 wherein the stack is bonded to an acoustic damper at one end of the stack.

33. Apparatus according to claim 30 wherein the piezoelectric material or materials from which the vibrators and isolation sections are formed have substantially same acoustic impedance.

34. Apparatus according to claim 30 wherein the stack is mounted in a housing formed from a conducting material.

35. Apparatus according to claim 10 wherein the plurality of carrier frequencies comprises a pair of frequencies that straddle a range of frequencies for which bone tissue typically attenuates ultrasound by about 3 dB.

36. Apparatus according to claim 35 wherein the lower frequency of the pair of frequencies is less than about 150 KHz.

37. Apparatus according to claim 35 wherein the lower frequency is substantially equal to about 125 KHz.

38. Apparatus according to claim 35 wherein the upper frequency of the pair of frequencies is greater than about 300 KHz.

39. Apparatus according to claim 35 wherein the upper frequency is substantially equal to about 750 KHz.

40. Apparatus according to claim 10 wherein the controller controls the first transducer to transmit pulses at different carrier frequencies at different times.

41. A method according to claim 1 wherein transmitting ultrasound pulses comprises transmitting pulses at different carrier frequencies at different times.

42. A method according to claim 7 wherein determining a characteristic time of a pulse waveform comprises:
determining a first time at which the waveform is first detected;
determining a second time at which a first subsequent zero crossing of the waveform occurs; and
determining a difference between the first and second times.

43. A method according to claim 8 wherein a characteristic time of a pulse waveform is determined by a method comprising:
determining a first time at which the waveform is first detected;
determining a second time at which a first subsequent zero crossing of the waveform occurs; and
determining a difference between the first and second times.

44. Apparatus according to claim 15 wherein the characteristic time period of the waveform of a pulse is a time period between a time at which the pulse is first detected by the at least one second transducer and a first subsequent zero crossing of the pulse pressure detected by the transducer.

45. Apparatus according to claim 16 wherein the characteristic time period of the waveform of the pulse is a time period between a time at which the pulse is first detected by the at least one second transducer and a first subsequent zero crossing of the pulse pressure detected by the transducer.

46. Apparatus according to claim 17 wherein the characteristic time period of the waveform of the pulse is a time period between a time at which the pulse is first detected by the at least one second transducer and a first subsequent zero crossing of the pulse pressure detected by the transducer.

47. Apparatus according to claim 20 wherein the characteristic time period of the waveform of the pulse is a time period between a time at which the pulse is first detected by the at least one second transducer and a first subsequent zero crossing of the pulse pressure detected by the transducer.
